(19) United States
(12) Patent Application Publication
(10) Pub. No.: US 2025/0186047 A1
(43) Pub. Date: Jun. 12, 2025

(54) DEVICES AND METHODS FOR TREATMENT OF FISTULAE

(71) Applicant: Cook Biotech Incorporated, West Lafayette, IN (US)

(72) Inventor: Bhavin Shah, West Lafayette, IN (US)

(73) Assignee: Cook Biotech Incorporated, West Lafayette, IN (US)

(21) Appl. No.: 18/966,698

(22) Filed: Dec. 3, 2024

Related U.S. Application Data
(60) Provisional application No. 63/607,731, filed on Dec. 8, 2023.

(51) Int. Cl. A61B 17/12; A61B 17/00

(52) U.S. Cl. CPC A61B 17/12031; A61B 2017/00641

(57) ABSTRACT

The present disclosure provides medical devices and methods for using said medical devices for treating a fistula in a patient. In some aspects the medical devices may include a plug body configured for receipt within the fistula tract, and an elongate member attached to a first location along the length of the plug body and configured to form a looped assembly extending through the fistula tract and along the path, wherein the looped assembly includes a length of the plug body that is free of the elongate member. In other aspects the medical devices may include a plug body having a longitudinal core in which portions of a plurality of biocompatible sheets are fixed to one another and also having a plurality of fin portions positioned or positionable to extend radially outward from the core in different directions.

### BACKGROUND

The present invention relates generally to medical technology and in particular aspects to methods and systems for addressing fistulae and other passageways and openings in the body.

As further background, there exist a variety of passageways and openings in the body which can be plugged or otherwise occupied by medical implants and materials to provide benefit to the patient. For example, it may be desirable to plug or otherwise treat a fistula. A variety of fistulae can occur in humans. These fistulae can occur for a variety of reasons, such as but not limited to, as a congenital defect, as a result of inflammatory bowel disease, such as Crohn's disease, irradiation, trauma, such as childbirth, or as a side effect from a surgical procedure.

Anorectal fistulae can result from infection in the anal glands, which are located around the circumference of the distal anal canal that forms the anatomic landmark known as the dentate line. Approximately 20-40 such glands are found in humans. Infection in an anal gland can result in an abscess. This abscess then can track through soft tissues (e.g., through or around the sphincter muscles) into the perianal skin, where it drains either spontaneously or surgically. The resulting void through soft tissue is known as a fistula. The internal or inner opening of the fistula, usually located at or near the dentate line, is known as the primary opening. Any external or outer openings, which are usually located in the perianal skin, are known as secondary openings.

A rectovaginal fistula is a fistula opening that develops between the vagina and the rectum. Rectovaginal fistula can result from injury during childbirth, inflammatory bowel disease such as Crohn's disease, radiation treatment or other cancer in the pelvic area, complication after surgery, and/or diverticulitis.

There remain needs for improved and/or alternative devices and methods for addressing fistulae and other passageways and openings in the body. The present invention is addressed to those needs.

### SUMMARY

In some aspects, provided are devices and systems useful for treating a wound or other opening. In accordance with some forms of the disclosure, such devices are useful for the treatment of a fistula. Accordingly, in one embodiment the present disclosure provides a medical device for treating a fistula in a patient, the fistula having a fistula tract extending between a primary opening and a secondary opening, the patient having a volume of tissue around which extends a path between the primary opening and the secondary opening. The medical device comprises a plug body configured for receipt within the fistula tract, and an elongate member attached to a first location along the length of the plug body and configured to form a looped assembly extending through the fistula tract and along the path, wherein the looped assembly includes a length of the plug body that is free of the elongate member and/or free of support against tension by the elongate member. In certain embodiments, the elongate member comprises a first segment configured to span the path between the primary opening and the second opening. In some forms, the plug body comprises an enlarged end having a diameter greater than a diameter of the plug body at a central portion. In some forms, the plug body comprises a sealing member configured to contact patient tissue around the primary opening. In accordance with certain embodiments, the plug body comprises an absorbable material, for example an extracellular matrix material and/or a synthetic material. In certain embodiments, the elongate member comprises a biodegradable material. In some forms, the biodegradable material is configured to persist for at least 30 days after implantation. The elongate member may comprise a looped portion, and wherein a portion of the plug body is configured to engage the looped portion. In accordance with some forms, an attachment member is attached to a second location along the length of the plug body, the second location spaced from the first location, and wherein the attachment member is configured to engage the elongate member to form the looped assembly. In some forms, the attachment member and the elongate member comprise biodegradable suture material.

In another embodiment, the present disclosure provides a method of treating a fistula, the fistula having a fistula tract extending between a primary opening and a secondary opening, the patient having a volume of tissue around which extends a path between the primary opening and the secondary opening, the method comprising: inserting a plug body into the fistula, wherein an elongate member is attached to a first location along the length of the plug body, and securing the plug body within the fistula by forming a looped assembly with the plug body and the elongate member, the looped assembly extending through the fistula tract and along the path, wherein the looped assembly includes a length of the plug body that is free of the elongate member and/or free of support against tension by the elongate member. Some forms of practicing the method comprise pulling the plug body into the fistula tract using the elongate member. In certain embodiments, the plug body comprises an enlarged end having a diameter greater than a diameter of the plug body at a central portion, and wherein the enlarged end is configured to become wedged into the primary opening upon inserting. The method may also comprise positioning a sealing member against patient tissue around the primary opening. In certain embodiments, the plug body comprises an absorbable material, for example an extracellular matrix material and/or a synthetic material. In some forms, the elongate member comprises a biodegradable material. In accordance with some forms, the biodegradable material is configured to persist for at least 30 days after implantation. In certain embodiments, the elongate member comprises a looped portion, and wherein the device is secured by attaching a distal end of the looped portion to the plug body.

In another embodiment, the present disclosure provides a medical device for treating a fistula in a patient, the medical device comprising a plug body configured for receipt within a fistula tract of the fistula. The plug body comprising a plurality of sheets of biocompatible material. The plug body includes a longitudinally extending core in which a first set of longitudinally extending portions of the sheets are fixed relative to one another. The plug body further includes a plurality of radial fin portions provided by a second set of longitudinally extending portions of the sheets that are movable relative to one another and positioned or are positionable to extend radially outwardly in different directions from the longitudinally extending core of the plug body. In accordance with some forms, the sheets are positioned in a stacked configuration, and the longitudinally extending core comprises suture material affixing the first set of longitudinally extending portions of the sheets to one another. In certain embodiments, the suture material includes at least one suture line comprising a suture extending from a leading end of the plug body to a trailing end of the plug body. In some forms, longitudinally extending side edges of the radial fin portions define a tapering width of the plug body. The radial fin portions may have serrated longitudinally extending side edges. In certain embodiments, the plurality of sheets comprises 2 to 6 sheets. The sheet may comprise at least 2 layers of biocompatible material. In accordance with some forms, a first suture portion extends beyond a leading end of the plug body, optionally wherein the first suture portion comprises a looped suture portion. In some embodiments, a second suture portion extends beyond a trailing end of the plug body, optionally wherein the second suture portion comprises a needle attached to the second suture portion. The first and second suture portions may be configured to extend around a volume of tissue around which extends a path between a primary opening and a secondary opening of the fistula.

Still further embodiments, as well as features and advantages of embodiments described herein, will be apparent to persons skilled in the relevant field from the descriptions herein.

### BRIEF DESCRIPTION OF THE DRAWINGS

FIG. 1 provides a perspective view of one embodiment of a medical device as disclosed herein.

FIG. 2 provides a perspective view of one embodiment of a medical device as disclosed herein.

FIG. 3A provides a cross-sectional view of an anal fistula, and a medical device as disclosed herein positioned within the anal fistula.

FIG. 3B provides a cross-sectional view of an anal fistula, and a medical device as disclosed herein positioned within the anal fistula.

FIG. 4A provides a cross-sectional view of a recto-vaginal fistula, and a medical device as disclosed herein positioned within the recto-vaginal fistula.

FIG. 4B provides a cross-sectional view of a recto-vaginal fistula, and a medical device as disclosed herein positioned within the recto-vaginal fistula.

FIG. 5 provides a perspective side view of one embodiment of a medical device as disclosed herein.

FIG. 6 provides a perspective top-down view of one embodiment of a medical device as disclosed herein.

### DETAILED DESCRIPTION

Reference will now be made to certain embodiments, some of which are illustrated in the drawings, and specific language will be used to describe the same. It will nevertheless be understood that no limitation of the scope of the disclosure is thereby intended. Any alterations and further modifications in the described embodiments and any further applications of the principles as described herein are contemplated as would normally occur to one skilled in the art to which this disclosure relates.

As disclosed above, aspects of the present disclosure relate to devices useful for treating a fistula in a patient. Implantation and fixation of a scaffold or plug in an anal or recto-vaginal fistula is very challenging. It is also difficult to get good purchase of the tissue to suture and secure the plug after implantation. Devices and methods of the present disclosure can eliminate the need to suture the device within the rectum, saving time, cost, and reducing patient discomfort. In some aspects, the present disclosure provides methods of using and/or preparing such devices. In accordance with certain embodiments, the present disclosure provides a medical device for treating a fistula having a fistula tract extending between a primary opening and a secondary opening, the patient having a volume of tissue around which extends a path between the primary opening and the secondary opening.

Devices of the present disclosure may include a plug body. In certain embodiments the plug body is configured to extend the length of a fistula tract, from the primary opening to the secondary opening. In some forms the plug body has a length from a first end to a second end of 0.5 cm to 20 cm, preferably 0.75 cm to 15 cm, more preferably 1 cm to 10 cm. The plug body may be configured to fill the fistula tract. The plug body may have a diameter of 2 mm to 20 mm, preferably 2 mm to 10 mm. In accordance with some forms, the plug body may have a generally constant diameter extending from a first end to a second end. In other embodiments, the plug body may have a variable diameter. For example, in certain embodiments, the plug body may have a larger diameter at the second end than the first end. In this way, the first end is configured as a leading end when pulled into the fistula tract. The enlarged second end may be configured to wedge into the fistula tract at or near the primary opening.

In accordance with some forms, a plug body may comprise a sheet form material. For example, in certain embodiments the plug body comprises a plurality of sheets of a biocompatible material. The biocompatible material may comprise an absorbable material. In accordance with some forms, the absorbable material comprises an extracellular matrix sheet material and/or a biocompatible synthetic material. For example, in certain embodiments the plug body comprises one or more sheets of an extracellular matrix material and one or more sheets of a biocompatible synthetic material. In some forms, the biocompatible synthetic material comprises an absorbable polymer sheet, preferably wherein the absorbable polymer sheet is porous. In some forms, the absorbable polymer sheet comprises an electrospun material. Each of the individual sheets of biocompatible material may comprise one or more layers of biocompatible material. For example, an extracellular matrix sheet material may comprise 1 or more layers of extracellular matrix material, preferably 2 or more layers of extracellular matrix material, preferably 2 to 8 layers of extracellular matrix material, more preferably 2 to 4 layers of extracellular matrix material. The individual layers of the biocompatible material may be bonded together by any suitable technique to form the biocompatible sheets.

The plug body may also include a longitudinally extending core in which a first set of longitudinally extending portions of the sheets are fixed relative to one another. Thus, the plug body may comprise a plurality of biocompatible material sheets forming a stacked configuration and a suture securing the biocompatible material sheets together. Such a configuration is advantageous as the load bearing member (e.g. the core) extends the length of the plug body, and thus can provide the user the tensile strength needed to pull the device into a fistula tract. The plug body may further include a plurality of radial fin portions provided by a second set of longitudinally extending portions of the sheets that are movable relative to one another and positioned or positionable to extend radially outwardly in different directions from the longitudinally extending core of the plug body.

In certain embodiments, the longitudinally extending core comprises a suture material. The suture material may include a single length of suture, or multiple lengths of suture, and may form any suitable stitching pattern that fixes longitudinally extending portions of the sheet material together, for example a lock-stitch pattern or a running straight pattern. The suture material may extend at least the length of the plug body, from a leading end to a trailing end. In certain embodiments, a single suture forms a suture line extending the length of the plug body, and such suture line can extend in a generally straight path (e.g. extending parallel to a longitudinal axis of the plug body) or in a non-straight path (e.g. extending in a waveform path, such as a zig-zag or sine wave path). In some forms, the suture(s) extend(s) beyond the end(s) of the biocompatible material. The suture can be used to position or secure a plug body into a fistula tract (e.g. by pulling the plug body through the fistula tract). In some forms, the suture comprises a first suture portion, extending from the leading end of the plug body, and a second suture portion extending from the trailing end of the plug body. In certain embodiments, the first suture portion of the suture comprises a looped suture portion to facilitate grasping of the suture and pulling of the plug body through a fistula tract. In certain embodiments, the second suture portion of the suture may be attached to a needle for facilitating securing of the plug body at the internal fistula opening. In accordance with some forms, the plug body comprise a single suture which forms the first suture portion, extends the length of the plug body, and forms the second suture portion. The longitudinally extending core may also comprise one or more discrete suture segments comprising an interrupted suture line having one or more longitudinal portions of the plug body free of suture material. The needle may be crimped or otherwise attached to the suture. In some forms, the sheets comprise a generally planar material having a rectangular shape. The shape of the sheet material may also be tapered such that longitudinally extending side edges of the radial fin portions define a tapering width of the plug body. For example, in some forms the width of the leading end of some of or each of the sheets, and thus in some forms the shape of the stack as a whole, is narrowed from side edge to side edge as compared to the width of the trailing end from side edge to side edge. The side edges of the sheets may be linear, curvilinear, or serrated. In certain embodiments the side edges of the sheets, forming the outermost radially projecting edges of the radial fin portions, comprise a serrated edge, which may allow for better filling of complex fistula passages and allow for better immobilization of the graft through the fistula passage. In some forms, the sheets may be fanned out (i.e. positioned so as to be extending radially from the longitudinally extending core in different directions) to form a three-dimensional profile. Knots or other enlarged profile retaining members may be positioned along the length of the suture, for example at point(s) where the suture exits the biocompatible sheet material at or near the leading end and preferably also the trailing end of the plug body. Such knots or retaining members may be positioned to immobilize the sheets along the length of the suture so as to prevent the sheets from moving longitudinally along the suture when pulling through the fistula tract. In accordance with some forms, the first and/or second end portion(s) of the suture that extend beyond the plug body is/are of sufficient length so as to extend around a volume of tissue around which extends a path between the primary opening and the secondary opening, such that the first and seconds ends may be secured together to secure the plug body in place within the fistula tract.

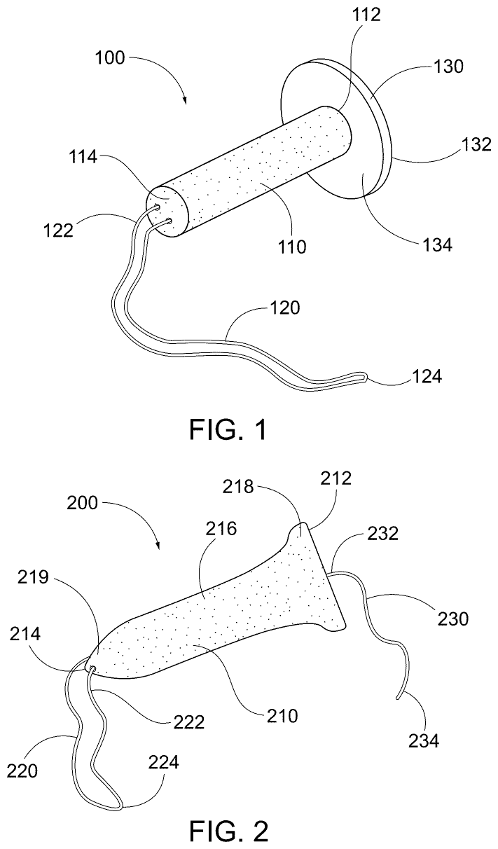

With reference now to the embodiment illustrated in FIG. 1, shown is one embodiment of a medical device for treating a fistula in a patient. Medical device 100 comprises plug body 110 and elongate member 120. In the illustrated embodiment, plug body 110 comprises a cylindrical body extending from first end 112 to second end 114. Elongate member 120 is shown extending from second end 114. In alternative embodiments, elongate member 120 may extend from near second end 114. Elongate member 120 extends from a proximal end 122 attached to plug body 110, to distal end 124. The illustrated embodiment further comprises capping member 130. In some forms capping member 130 is affixed to first end 112 of plug body 110. Capping member 130 comprises a first side 132 opposing a second side 134 and has a width greater than a maximum width of the plug body. Elongate member 120 is shown as a looped member. Such a configuration may be advantageous, for example, to loop around and secure to a capping member.

In certain embodiments, medical devices of the present disclosure comprise a capping member. The capping member may be configured to seal a fistula opening, for example the primary and/or secondary fistula opening. Such capping members may comprise a smooth outer surface configured to allow passage of material (e.g. fecal matter) over the surface of the cap without obstruction. The inner surface of the capping member may be configured for contact with body tissue, for example intestinal, vaginal, or epidermal tissue. In some forms, the capping member has a width greater than a width of the plug body. The capping member may have any suitable shape, for example the capping member may be circular, ovoid, or disc shaped.

FIG. 2 illustrates another embodiment of a medical device for treating a fistula in a patient. Medical device 200 comprises plug body 210 and elongate member 220. In the illustrated embodiment, plug body 210 comprises a cylindrical body portion 216 extending from first end 212 to second end 214. Plug body 210 comprises a flared portion 218 near first end 212. When present a flared portion of a plug body has an increased diameter than an adjacent portion of the plug body. In this way the flared portion may operate as a plug to fill and/or seal a portion of a fistula tract, for example a primary or secondary opening. While the flared portion is illustrated at one end of plug body 210, it is within the scope of the disclosure to provide a plug body having a flared portion at or near both ends of the plug body. In some forms a flared portion may be positioned in a more central portion of the plug body. Plug body 210 comprises a tapered portion 219 near second end. When present a tapered portion of a plug body has a decreased diameter than an adjacent portion of the plug body. In this way, the tapered portion may operate as a leading end to position the medical device within the fistula trace. While the tapered portion is illustrated at one end of plug body 210, it is within the scope of the disclosure to provide a plug body having a tapered portion at or near both ends of the plug body. In some forms a tapered portion may be positioned in a more central portion of the plug body. Flared and/or tapered portions may comprise substantially the same material as the plug body. Alternatively, flared and/or tapered portions may comprise a material different than the material of the rest of the plug body. For example, tapered and/or elongate portions may be more or less dense than other portions of the plug body. Elongate member 220 is shown extending from second end 214. In alternative embodiments, elongate member 220 may extend from near second end 214. Elongate member 220 extends from a proximal end 222 attached to plug body 210, to distal end 224. In the illustrated embodiment, attachment member 230 extends from first end 212 having a proximal portion 232 and a distal portion 234. In some forms, an attachment member is configured to attach to the elongate member, for example at distal end 224, to secure the medical device within the fistula. Elongate member 220 is shown as a looped member. Such a configuration may be advantageous, for example, to facilitate securing the attachment member to the elongate member.

Elongate member and/or attachment member may comprise any suitable material. In accordance with some forms, the elongate member and/or attachment member comprises a suture material. The suture material may be biodegradable or nonbiodegradable. Suitable synthetic materials that may be used in the presently disclosed device include, but are not limited to, polygalactin, polydioxanone, hyaluronic acid, polyglycolic acid, and polyethylene terephthalate. Further biodegradable polymers which may be used include, but are not limited to, poly(L-lactic acid), polycaprolactone, poly(lactide-co-glycolide), poly(hydroxybutyrate), poly(hydroxybutyrate-co-valerate), polydioxanone, polyorthoester, polyanhydride, poly(glycolic acid), poly(D,L-lactic acid), poly(glycolic acid-co-trimethylene carbonate), polyhydroxyalkanates, polyphosphoester, polyphosphoester urethane, poly(amino acids), cyanoacrylates, polyester urea, poly(trimethylene carbonate), poly(iminocarbonate), copoly(ether-esters) (e.g., PEO/PLA), polyalkylene oxalates, and polyphosphazenes. These or other biodegradable materials may be used, for example, where only a temporary function is desired, and/or in combination with non-biodegradable materials where only a temporary participation by the biodegradable material is desired. Non-biodegradable, or biostable polymers that may be used include, but are not limited to, polytetrafluoroethylene (PTFE) (including expanded PTFE), polyethylene terephthalate (PET), polyurethanes, silicones, and polyesters and other polymers such as, but not limited to, polyolefins, polyisobutylene and ethylene-alphaolefin copolymers; acrylic polymers and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins, polycarbonates; polyoxymethylenes; polyimides; polyethers; epoxy resins, polyurethanes; rayon; and rayon-triacetate.

One or more of the elongate member and/or attachment member may comprise a looped portion. One or more of the elongate member and/or attachment member may comprise 2 or more lengths of material (e.g. suture material). In preferred forms, the combined length of the elongate member and/or attachment member, when present, is sufficient to extend from the primary opening to the secondary opening, external of the fistula tract, to form a looped seton around a volume of patient tissue.

While the illustrated plug bodies are generally cylindrical, such plugs and other implants when utilized in the presently disclosed device can be shaped and configured in a variety of manners. These include various shaped, three-dimensional constructs, and even some sheet-like or generally two-dimensional implantable materials. When generally cylindrical, a plug body portion can, for example, have a diameter of about 0.5 mm to about 30.0 mm and a length of about 0.5 cm to about 30 cm, although larger or smaller values for these dimensions could be used in accordance with the invention. Thus, a medical device in some forms of the invention will include a plug body that is generally cylindrical, and has a diameter ranging from about 1.0 mm to about 18.0 mm, or from about 3.0 mm to about 12.0 mm, or from about 4.0 mm to about 8.0 mm, and a length ranging from about 2.0 cm to about 18.0 cm, or from about 4.0 cm to about 12.0 cm. As well, a plug body may be formed with one or more of a variety of biocompatible materials including some that are naturally derived and some that are non-naturally derived, as described elsewhere herein.

The plug body may have a constant or varying cross-sectional area along its length. Illustratively, a plug body may exhibit a generally cylindrical shape, a conical shape or any other suitable shape including some that have tapered and/or non-tapered longitudinal portions. As well, a cross section of a particular portion of a plug body may exhibit a variety shapes including some that have rectilinear and/or curvilinear features. Thus, a plug body can include a portion having a generally circular or non-circular (e.g., elliptical, square, star-shaped, hexagonal, etc.) cross section. Additionally or alternatively, a plug body can include various other three-dimensional volumetric body portions such as but not limited to braids, tubes, hemi-cylinders, strands, threads, strips, pieces, slabs, wedges, blocks and other shaped body portions having suitable dimensions.

The plug bodies described herein can be formed in any suitable manner including but not limited to by extrusion, using a mold or form, construction around a mandrel, and/or combinations or variations thereof. In some embodiments, a plug body is formed with a reconstituted or otherwise reassembled ECM material. Plug bodies may also be formed by folding or rolling, or otherwise overlaying one or more portions of one or more biocompatible materials, such as one or more layers of a biocompatible sheet material. The overlaid biocompatible sheet material can be compressed and dried or otherwise bonded into a volumetric shape such that a substantially unitary construct is formed. In some forms, an inventive implant component is constructed by randomly or regularly packing one or more pieces of single or multilayer ECM sheet material within a mold and thereafter processing the packed material.

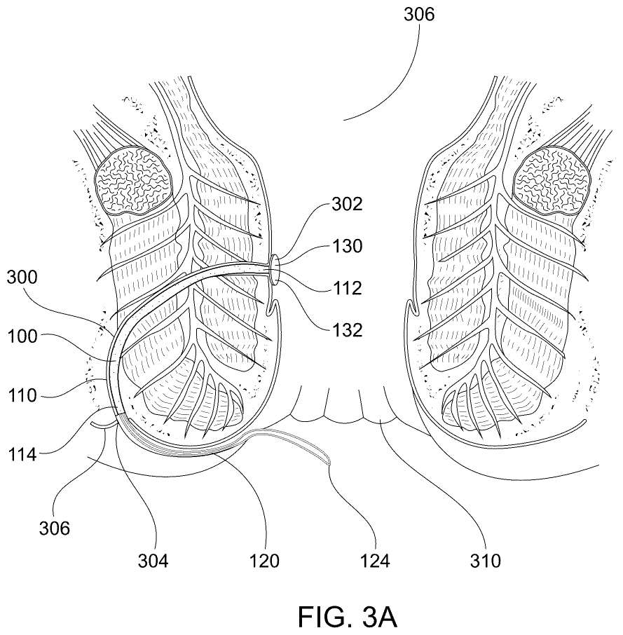

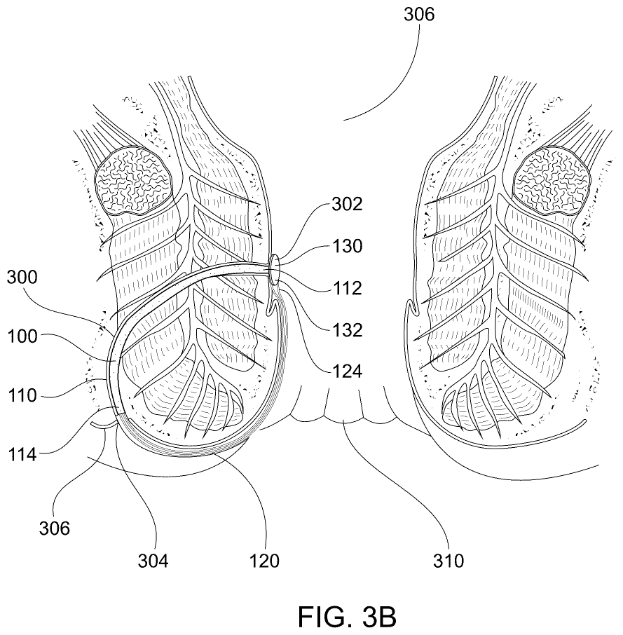

Turning now to the embodiment illustrated in FIGS. 3A and 3b, a cross-sectional view of an anal fistula 300 extending from primary opening 302 and secondary opening 304. Primary opening 302 opens to anal canal 306. Secondary opening opens to perianal skin 308. Device 100 is positioned within anal fistula 300. In the illustrated embodiment, plug body 110 extends from primary opening 302 to secondary opening 304. As shown in FIG. 3B, elongate member 120 is passed through anus 310 and is secured to capping member 120.

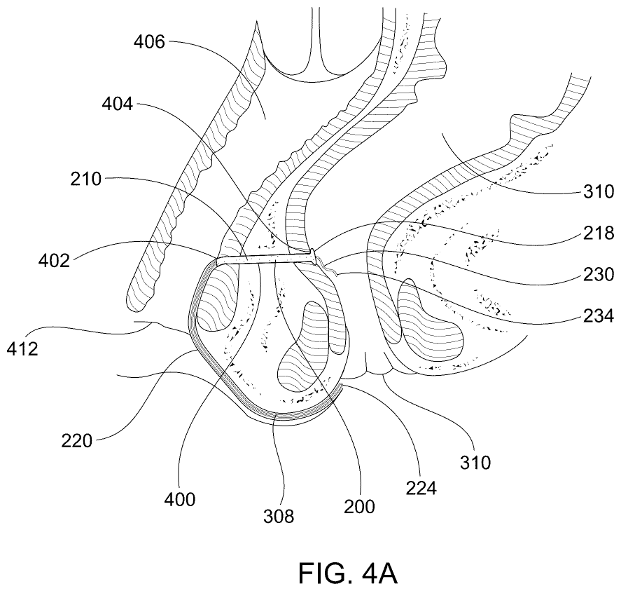

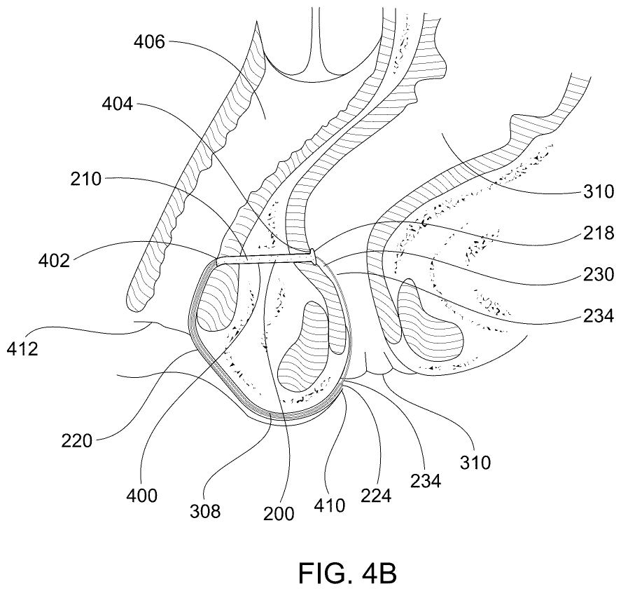

FIGS. 4A and 4B show a cross-sectional view of a recto-vaginal fistula 400 extending from vaginal opening 402 to anal opening 404. vaginal fistula opening 402 opens into vaginal canal 406. Anal fistula opening 404 opens into anal canal 310. In the illustrated embodiment, plug body 210 extends from anal fistula opening 404 to vaginal fistula opening 402. As shown in FIG. 4B, elongate member 220 is passed through the vaginal opening 412 and over perianal skin 308. In the illustrated embodiment, attachment member 230 extends out of anus 310 such that attachment point 410, between distal end 224 of elongate member 220 and distal portion 234 of attachment member 230. The lengths of the elongate member and the attachment member may be adjust so as to position the attachment point at a desired location, e.g within the vaginal canal or anal canal, or exterior of the body.

In use, any of the medical devices discussed herein may be attached to a probe. For example, in some forms the present disclosure provides methods of treating a fistula comprising passing a probe through a fistula, attaching a medical device as described herein to the distal end of the probe, and pulling the plug body into the fistula. Such pulling may be achieved by retracting the probe back through the fistula. Such methods may further comprise securing the medical device in place. In accordance with some forms, the device is secured by forming a looped assembly with the plug body and the elongate member. In certain embodiments, the looped assembly extends through the fistula tract and along a path between the primary opening and the secondary opening. Of course, it will be appreciated that the devices according to the present invention may be used by the person skilled in the art to treat simple anal and recto-vaginal fistulas using variations of the techniques exemplified above. For instance, when used to treat a recto-vaginal fistula, the probe may be thread through the recto-vaginal fistula from the rectal opening or the vaginal opening. Similarly, it will be appreciated that while uses of the devices according to the present invention have been described above in relation to simple anal and recto-vaginal fistulas, the person skilled in the art will understand that the devices may also find application in many other types of fistula. The devices of the present invention find particular application in fistulas adjacent to which there is a substantial body of tissue about which the device may be affixed. Preferably the fistulas to be treated comprise or are located near at least one external opening such that the device lies partially outside of the body. That said however, the use of the devices of the present invention entirely internally is also envisaged.

As discussed herein, medical devices of the present disclosure may be configured to form a looped assembly. Such looped assemblies may comprise a plug body, one or more elongate members, and/or one or more attachment members. In accordance with some forms, the looped assembly may contain a length of the plug body that is free of the elongate member, and/or the biocompatible sheet material of the plug body, when tension is applied to the looped assembly, may receive the tension. In certain embodiments, the length of the plug body that is free of the elongate member comprises a portion of the longitudinal length of the biocompatible sheet material of the plug. In accordance with some forms, a substantial portion of the longitudinal length of the biocompatible sheet material (e.g. at least about 70% thereof, or at least about 80% thereof) receives tension applied to the looped assembly and/or such tension may cause the biocompatible sheet material of the plug body to stretch or otherwise deform.

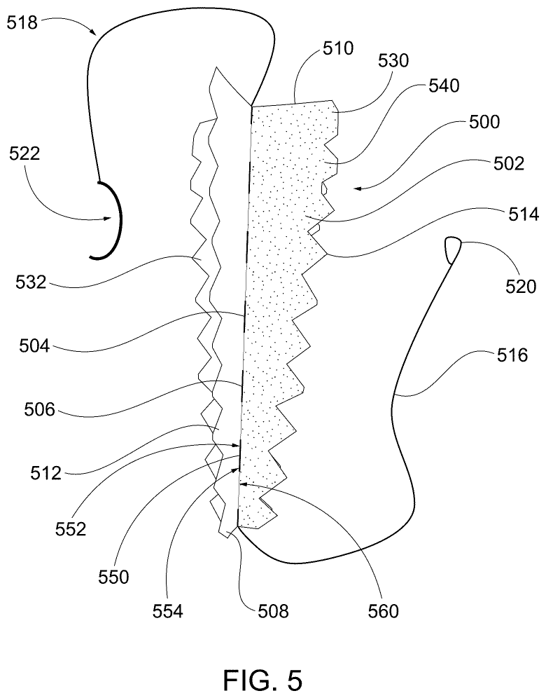

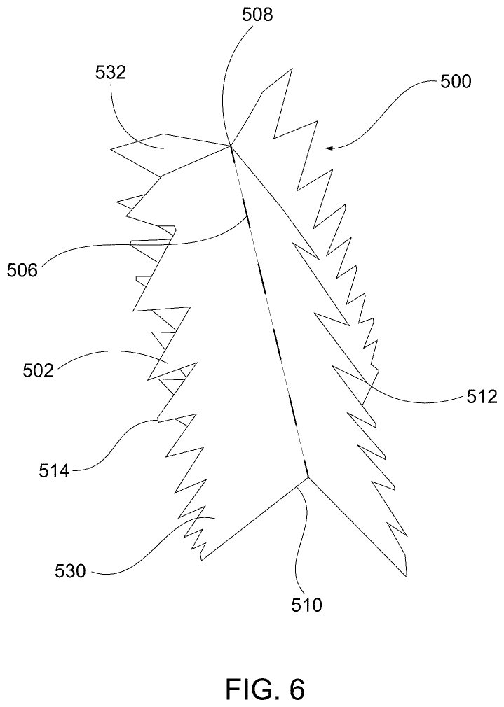

With reference to the embodiments shown in FIGS. 5 and 6, shown is plug body 500, comprising biocompatible sheet material 502. Plug body 500 includes longitudinally extending core 504 comprising suture 506. As detailed above, the longitudinally extending core may comprise a single suture line extending the length of the longitudinally extending core. Alternatively, the longitudinally extending core may comprise or more discrete suture segments 550 spaced along the length of the longitudinally extending core. Discrete suture segments 550 comprise a length of suture extending from a first end 552 to a second end 554. Generally, ends 552 and 554 are affixed to the biocompatible sheet material, for example stitched thereto. In some forms, a suture free portion 560 extends between adjacent ones of discrete suture segments. The illustrated embodiments comprise a first sheet 530 and a second sheet 532. As discussed above, a plug body may comprise additional sheets. The sheet material 502 extends from leading end 508 to trailing end 510. In the illustrated embodiment, the plug body is tapered such that trailing end 510 is wider than leading end 508. Side edges 512, 514 are serrated. First suture portion 516 extends from leading end 508, and includes loop 520. Second suture portion 518 extends from trailing end 510 and includes needle 522. In the illustrated embodiment needle 522 is shown as a curved needle, such a needle shape may facilitate securing the suture to patient tissue although any suitable needle shape may be incorporated. In accordance with some forms, the biocompatible sheet material may comprise perforations 540. Such perforations, or holes, may facilitate fluid flow and enhance draining of fluid from the fistula.

In certain embodiments, one or more device components of the medical device, for example the biocompatible sheet material, will be comprised of a remodelable material. Particular advantage can be provided by devices that incorporate a remodelable collagenous material. Such remodelable collagenous materials, whether reconstituted or naturally-derived, can be provided, for example, by collagenous materials isolated from a warm-blooded vertebrate, and especially a mammal. Such isolated collagenous material can be processed so as to have remodelable, angiogenic properties and promote cellular invasion and ingrowth. Remodelable materials may be used in this context to promote cellular growth on, around, and/or in bodily regions in which inventive devices are implanted or engrafted.

Suitable remodelable materials can be provided by collagenous extracellular matrix (ECM) materials possessing biotropic properties. For example, suitable collagenous materials include ECM materials such as those comprising submucosa, renal capsule membrane, dermal collagen, dura mater, pericardium, fascia lata, serosa, peritoneum or basement membrane layers, including liver basement membrane. Suitable submucosa materials for these purposes include, for instance, intestinal submucosa including small intestinal submucosa, stomach submucosa, urinary bladder submucosa, and uterine submucosa. Collagenous matrices comprising submucosa (potentially along with other associated tissues) useful in the present invention can be obtained by harvesting such tissue sources and delaminating the submucosa-containing matrix from smooth muscle layers, mucosal layers, and/or other layers occurring in the tissue source. For additional information as to some of the materials useful in the present invention, and their isolation and treatment, reference can be made, for example, to U.S. Pat. Nos. 4,902,508, 5,554,389, 5,993,844, 6,206,931, and 6,099,567.

Submucosa-containing or other ECM tissue used in the invention is preferably highly purified, for example, as described in U.S. Pat. No. 6,206,931 to Cook et al. Thus, preferred ECM material will exhibit an endotoxin level of less than about 12 endotoxin units (EU) per gram, more preferably less than about 5 EU per gram, and most preferably less than about 1 EU per gram. As additional preferences, the submucosa or other ECM material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plaque forming units (PFU) per gram, more preferably less than about 5 PFU per gram. These and additional properties of submucosa or other ECM tissue taught in U.S. Pat. No. 6,206,931 may be characteristic of any ECM tissue used in the present invention.

A typical layer thickness for an as-isolated submucosa or other ECM tissue layer used in the invention ranges from about 50 to about 250 microns when fully hydrated, more typically from about 50 to about 200 microns when fully hydrated, although isolated layers having other thicknesses may also be obtained and used. These layer thicknesses may vary with the type and age of the animal used as the tissue source. As well, these layer thicknesses may vary with the source of the tissue obtained from the animal source.

Suitable bioactive agents may include one or more bioactive agents native to the source of the ECM tissue material. For example, a submucosa or other remodelable ECM tissue material may retain one or more growth factors such as but not limited to basic fibroblast growth factor (FGF-2), transforming growth factor beta (TGF-beta), epidermal growth factor (EGF), cartilage derived growth factor (CDGF), and/or platelet derived growth factor (PDGF). As well, submucosa or other ECM materials when used in the invention may retain other native bioactive agents such as but not limited to proteins, glycoproteins, proteoglycans, and glycosaminoglycans. For example, ECM materials may include heparin, heparin sulfate, hyaluronic acid, fibronectin, cytokines, and the like. Thus, generally speaking, a submucosa or other ECM material may retain one or more bioactive components that induce, directly or indirectly, a cellular response such as a change in cell morphology, proliferation, growth, protein or gene expression.

Submucosa-containing or other ECM materials of the present invention can be derived from any suitable organ or other tissue source, usually sources containing connective tissues. The ECM materials processed for use in the invention will typically include abundant collagen, most commonly being constituted at least about 80% by weight collagen on a dry weight basis. Such naturally-derived ECM materials will for the most part include collagen fibers that are non-randomly oriented, for instance occurring as generally uniaxial or multi-axial but regularly oriented fibers. When processed to retain native bioactive factors, the ECM material can retain these factors interspersed as solids between, upon and/or within the collagen fibers. Particularly desirable naturally-derived ECM materials for use in the invention will include significant amounts of such interspersed, non-collagenous solids that are readily ascertainable under light microscopic examination with appropriate staining. Such non-collagenous solids can constitute a significant percentage of the dry weight of the ECM material in certain inventive embodiments, for example at least about 1%, at least about 3%, and at least about 5% by weight in various embodiments of the invention.

The submucosa-containing or other ECM material used in the present invention may also exhibit an angiogenic character and thus be effective to induce angiogenesis in a host engrafted with the material. In this regard, angiogenesis is the process through which the body makes new blood vessels to generate increased blood supply to tissues. Thus, angiogenic materials, when contacted with host tissues, promote or encourage the formation of new blood vessels into the materials. Methods for measuring in vivo angiogenesis in response to biomaterial implantation have recently been developed. For example, one such method uses a subcutaneous implant model to determine the angiogenic character of a material. See, C. Heeschen et al., Nature Medicine 7 (2001), No. 7, 833-839. When combined with a fluorescence microangiography technique, this model can provide both quantitative and qualitative measures of angiogenesis into biomaterials. C. Johnson et al., Circulation Research 94 (2004), No. 2, 262-268.

Further, in addition or as an alternative to the inclusion of such native bioactive components, non-native bioactive components such as those synthetically produced by recombinant technology or other methods (e.g., genetic material such as DNA), may be incorporated into an ECM material. These non-native bioactive components may be naturally-derived or recombinantly produced proteins that correspond to those natively occurring in an ECM tissue, but perhaps of a different species. These non-native bioactive components may also be drug substances. Illustrative drug substances that may be added to materials include, for example, anti-clotting agents, e.g. heparin, antibiotics, anti-inflammatory agents, thrombus-promoting substances such as blood clotting factors, e.g., thrombin, fibrinogen, and the like, and anti-proliferative agents, e.g. taxol derivatives such as paclitaxel. Such non-native bioactive components can be incorporated into and/or onto ECM material in any suitable manner, for example, by surface treatment (e.g., spraying) and/or impregnation (e.g., soaking), just to name a few. Also, these substances may be applied to the ECM material in a premanufacturing step, immediately prior to the procedure (e.g., by soaking the material in a solution containing a suitable antibiotic such as cefazolin), or during or after engraftment of the material in the patient.

Inventive devices can incorporate xenograft material (i.e., cross-species material, such as tissue material from a non-human donor to a human recipient), allograft material (i.e., interspecies material, with tissue material from a donor of the same species as the recipient), and/or autograft material (i.e., where the donor and the recipient are the same individual). Further, any exogenous bioactive substances incorporated into an ECM material may be from the same species of animal from which the ECM material was derived (e.g. autologous or allogenic relative to the ECM material) or may be from a different species from the ECM material source (xenogenic relative to the ECM material). In certain embodiments, ECM material will be xenogenic relative to the patient receiving the graft, and any added exogenous material(s) will be from the same species (e.g. autologous or allogenic) as the patient receiving the graft. Illustratively, human patients may be treated with xenogenic ECM materials (e.g. porcine-, bovine- or ovine-derived) that have been modified with exogenous human material(s) as described herein, those exogenous materials being naturally derived and/or recombinantly produced.

In certain forms, inventive devices include a material receptive to tissue ingrowth. Upon deployment of such devices in accordance with the present invention, cells from the patient can infiltrate the material, leading to, for example, new tissue growth on, around, and/or within the device. In some embodiments, the device comprises a remodelable material. In these embodiments, the remodelable material promotes and/or facilitates the formation of new tissue, and is capable of being broken down and replaced by new tissue. Remodelable ECM materials having a relatively more open matrix structure (i.e., higher porosity) are capable of exhibiting different material properties than those having a relatively more closed or collapsed matrix structure. For example, an ECM material having a relatively more open matrix structure is generally softer and more readily compliant to an implant site than one having a relatively more closed matrix structure. Also, the rate and amount of tissue growth in and/or around a remodelable material can be influenced by a number of factors, including the amount of open space available in the material's matrix structure for the infusion and support of a patient's tissue-forming components, such as fibroblasts. Therefore, a more open matrix structure can provide for quicker, and potentially more, growth of patient tissue in and/or around the remodelable material, which in turn, can lead to quicker remodeling of the material by patient tissue.

In this regard, any component of a medical graft product of the invention (including any ECM material) can have a level or degree of porosity. In certain embodiments, the porosity of a layer of ECM material is lowered by drying the material under compression. In general, compressing a pliable open matrix material, such as a pliable ECM material, increases the material's bulk density and decreases the material's porosity by decreasing the size of the voids in the open matrix. As is the case in certain aspects of the invention, when such a material is dried while being compressed, particularly under vacuum pressing conditions, the open matrix structure can become somewhat fixed in this relatively higher bulk density, lower porosity state (i.e., in a relatively more collapsed state). It should be noted that different compressing and drying techniques and/or methods, including different degrees of compressing and drying, can be designed through routine experimentation so as to allow for a material layer having an optimal degree of material bulk density and/or porosity for a particular application or procedure.

It is sometimes advantageous to perform drying operations under relatively mild temperature exposure conditions that minimize deleterious effects upon the ECM materials of the invention, for example native collagen structures and potentially bioactive substances present. Thus, drying operations conducted with no or substantially no duration of exposure to temperatures above human body temperature or slightly higher, say, no higher than about 38° C., will preferably be used in some forms of the present invention. These include, for example, vacuum pressing operations at less than about 38° C., forced air drying at less than about 38° C., or either of these processes with no active heating—at about room temperature (about 25° C.) or with cooling. Relatively low temperature conditions also, of course, include lyophilization conditions.

In additional embodiments, medical devices as disclosed herein can incorporate ECM tissue material that has been subjected to a process that expands the tissue material. In certain forms, such expanded materials can be formed by the controlled contact of an ECM material with a denaturing agent such as one or more alkaline substances until the material expands, and the isolation of the expanded material. Illustratively, the contacting can be sufficient to expand the ECM tissue material to at least 120% of (i.e. 1.2 times) its original bulk volume, or in some forms to at least about two times its original volume. Thereafter, the expanded material can optionally be isolated from the alkaline medium, e.g. by neutralization and/or rinsing. The collected, expanded material can be used in any suitable manner in the preparation of a material for administration to a patient. The expanded material can be enriched with bioactive components, comminuted, dried, and/or molded, etc., in the formation of an implantable body of a desired shape or configuration. In certain embodiments, a dried implant body formed with an expanded ECM tissue material can be compressible.

Treatment of an ECM tissue material with a denaturant, such as an alkaline material, can cause changes in the physical structure of the material that in turn cause it to expand. Such changes may include denaturation of the collagen in the material. In certain embodiments, it is preferred to expand the material to at least about three, at least about four, at least about 5, or at least about 6 or even more times its original bulk volume. It will be apparent to one skilled in the art that the magnitude of the expansion is related to several factors, including for instance the concentration or pH of the alkaline medium, the exposure time of the alkaline medium to the material, and temperature used in the treatment of the material to be expanded, among others. These factors can be varied through routine experimentation to achieve a material having the desired level of expansion, given the disclosures herein.

A collagen fibril is comprised of a quarter-staggered array of tropocollagen molecules. The tropocollagen molecules themselves are formed from three polypeptide chains linked together by covalent intramolecular bonds and hydrogen bonds to form a triple helix. Additionally, covalent intermolecular bonds are formed between different tropocollagen molecules within the collagen fibril. Frequently, multiple collagen fibrils assemble with one another to form collagen fibers. It is believed that the addition of an alkaline substance to the material as described herein can be conducted so as to not significantly disrupt the intramolecular and intermolecular bonds, but denature the material to an extent that provides to the material an increased processed thickness, e.g. at least twice the naturally-occurring thickness. ECM materials that can be processed to make expanded materials for use as substrates can include any of those disclosed herein or other suitable ECM's. Typical such ECM materials will include a network of collagen fibrils having naturally-occurring intramolecular cross links and naturally-occurring intermolecular cross links. Upon expansion processing as described herein, the naturally-occurring intramolecular cross links and naturally-occurring intermolecular cross links can be retained in the processed collagenous matrix material sufficiently to maintain the collagenous matrix material as an intact collagenous sheet material; however, collagen fibrils in the collagenous sheet material can be denatured, and the collagenous sheet material can have an alkaline-processed thickness that is greater than the thickness of the starting material, for example at least 120% of the original thickness, or at least twice the original thickness. The expanded ECM material can then be processed to provide foam or sponge substrates for use as or in the graft body, e.g. by comminuting, casting, and drying the processed material. Additional information concerning expanded ECM materials and their preparation is found in United States Patent Application Publication No. US20090326577 published Dec. 31, 2009, publishing U.S. patent application Ser. No. 12/489,199 filed Jun. 22, 2009, which is hereby incorporated herein by reference in its entirety.

Devices and methods of the present disclosure may be used to treat any suitable fistula. Devices and methods of the present disclosure may be used to treat anal fistula, including for example: anal, anorectal, intersphicteric, transphincteric, and/or suprasphincteric. Devices and methods of the present disclosure may be used to treat recto-vaginal fistula, including for example: anovulval, anovaginal, rectovulval, rectovaginal and/or rectovestibular fistula, wherein the recto-vaginal fistula may be classified anatomically as infrasphincteric, transphincteric, and/or suprasphincteric. Devices and methods of the present disclosure may be used to treat recto-prostatic fistula. Devices and methods of the present disclosure may be used to treat gastrointestinal fistula, including for example: trachea-oesophageal, gastro-cutaneous, ileo-cutaneous, colo-cutaneous, colo-vaginal and/or gastrointestinal-vascular fistula. Devices and methods of the present disclosure may be used to treat urinary fistula, including for example: urethrocutaneous, urethrovaginal, urethrovesical, vesciovaginal, rectovesical and/or rectourethral fistula. Devices and methods of the present disclosure may also be used to treat a fistula resulting from a body-piercing, or a skin-to-skin fistula. Typically said fistulas are complete (i.e. both ends open on a mucosal or exterior surface of the body). Complete fistulas may be external (i.e. between a hollow organ and an external surface of the body) or bimucosal (i.e. both ends open on a mucosal surface of the body).

The present invention also provides, in certain aspects, a line of medical products, wherein a medical product of the invention includes one or more devices, apparatuses or systems of the invention in a sealed package. In some forms of the invention, medical products are provided that include one or more inventive devices or systems enclosed within sterile medical packaging. Illustratively, such a medical product can have packaging including a backing layer and a front film layer that are joined by a boundary of pressure-adhesive as is conventional in medical packaging, wherein the contents of the packaging are sealed between the backing layer and front film layer. Sterilization of such a medical product may be achieved, for example, by irradiation, ethylene oxide gas, or any other suitable sterilization technique, and the materials and other properties of the medical packaging will be selected accordingly.

### EMBODIMENTS

The following provides an enumerated listing of some of the embodiments disclosed herein. It will be understood that this listing is non-limiting, and that individual features or combinations of features (e.g., 2, 3 or 4 features) as described in the Detailed Description above can be incorporated with the below-listed Embodiments to provide additional disclosed embodiments herein.

1. A medical device for treating a fistula in a patient, the fistula having a fistula tract extending between a primary opening and a secondary opening, the patient having a volume of tissue around which extends a path between the primary opening and the secondary opening, the medical device comprising:
a plug body configured for receipt within the fistula tract; and
an elongate member attached to a first location along the length of the plug body and configured to form a looped assembly extending through the fistula tract and along the path, wherein the looped assembly includes a length of the plug body that is free of the elongate member and/or free of support against tension by the elongate member.

2. The medical device of embodiment 1, wherein the elongate member comprises a first segment configured to span the path between the primary opening and the second opening.

3. The medical device of any one of the preceding embodiments, wherein the plug body comprises an enlarged end having a diameter greater than a diameter of the plug body at a central portion.

4. The medical device of any one of the preceding embodiments, wherein:
the plug body includes a sealing member configured to contact patient tissue around the primary opening.

5. The medical device of any one of the preceding embodiments, wherein the plug body comprises an absorbable material.

6. The medical device of embodiment 5, wherein the absorbable material comprises an extracellular matrix material.

7. The medical device of embodiment 5, wherein the absorbable material comprises a synthetic material.

8. The medical device of any one of the preceding embodiments, wherein the elongate member comprises a biodegradable material.

9. The medical device of embodiment 8, wherein the biodegradable material is configured to persist for at least 30 days after implantation.

10. The medical device of any one of the preceding embodiments, wherein the elongate member comprises a looped portion.

11. The medical device of embodiment 10, wherein a second location along the length of the plug body is configured to engage the looped portion.

12. The medical device of any one of embodiments 1-10, comprising an attachment member attached to a second location along the length of the plug body, the second location spaced from the first location, and wherein the attachment member is configured to engage the elongate member to form the looped assembly.

13. The medical device of embodiment 12, wherein the attachment member and the elongate member comprise biodegradable suture material.

14. A method of treating a fistula, the fistula having a fistula tract extending between a primary opening and a secondary opening, the patient having a volume of tissue around which extends a path between the primary opening and the secondary opening, the method comprising:
inserting a plug body into the fistula, wherein an elongate member is attached to a first location along the length of the plug body; and
securing the plug body within the fistula by forming a looped assembly with the plug body and the elongate member, the looped assembly extending through the fistula tract and along the path, wherein the looped assembly includes a length of the plug body that is free of the elongate member and/or free of support against tension by the elongate member.

15. The method of embodiment 14, wherein said inserting comprises pulling the plug body into the fistula tract using the elongate member.

16. The method of any one of embodiments 14 or 15, wherein the plug body comprises an enlarged end having a diameter greater than a diameter of the plug body at a central portion, and wherein said inserting causes the enlarged end to become wedged into the primary opening.

17. The method of any one of embodiments 14 through 16, comprising positioning a sealing member against patient tissue around the primary opening.

18. The method of any one of embodiments 14 through 17, wherein the plug body comprises an absorbable material.

19. The method of embodiment 18, wherein the absorbable material comprises an extracellular matrix material.

20. The method of embodiment 18, wherein the absorbable material comprises a synthetic material.

21. The method of any one of embodiments 14 through 20, wherein the elongate member comprises a biodegradable material.

22. The method of embodiment 21, wherein the biodegradable material is configured to persist for at least 30 days after implantation.

23. The method of any one of embodiments 14 through 22, wherein the elongate member comprises a looped portion.

24. A medical device for treating a fistula in a patient, the medical device comprising:
a plug body configured for receipt within a fistula tract of the fistula, the plug body comprising a plurality of sheets of biocompatible material, wherein the plug body includes a longitudinally extending core in which a first set of longitudinally extending portions of the sheets are fixed relative to one another, and wherein the plug body further includes a plurality of radial fin portions provided by a second set of longitudinally extending portions of the sheets that are movable relative to one another and positioned or positionable to extend radially outwardly in different directions from the longitudinally extending core of the plug body.

25. The medical device of embodiment 24, wherein:
the sheets are positioned in a stacked configuration; and
the longitudinally extending core comprises suture material affixing the first set of longitudinally extending portions of the sheets to one another.

26. The medical device of embodiment 25, wherein the suture material includes at least one suture line comprising a suture extending from a leading end of the plug body to a trailing end of the plug body.

27. The medical device of any one of embodiments 24 to 26, wherein longitudinally extending side edges of the radial fin portions define a tapering width of the plug body, optionally wherein the width of the plug body increases in a direction from a leading end of the plug body to a trailing end of the plug body.

28. The medical device of any one of embodiments 24 to 27, wherein the radial fin portions have serrated longitudinally extending side edges.

29. The medical device of any one of embodiments 24 to 28, wherein the plurality of sheets comprises only 2 to 6 sheets.

30. The medical device of any one of embodiments 24 to 29, wherein each of said sheets comprises at least 2 layers of biocompatible material.

31. The medical device of any one of embodiments 24 to 30, wherein the biocompatible material comprises an extracellular matrix material.

32. The medical device of any one of embodiments 24 to 31, wherein the biocompatible material comprises a porous absorbable polymer.

33. The medical device of any one of embodiments 24 to 32, comprising a first suture portion extending beyond a leading end of the plug body.

34. The medical device of embodiment 33, wherein the first suture portion comprises a looped suture portion.

35. The medical device of any one of embodiments 24 to 34, comprising a second suture portion extending beyond a trailing end of the plug body.

36. The medical device of embodiment 33, further comprising a needle attached to the second suture portion.

37. The medical device of any one of embodiments 33 to 35, wherein the first suture portion and the second suture portion are configured to extend around a volume of tissue around which extends a path between a primary opening and a secondary opening of the fistula.

38. A method of treating a fistula comprising:
positioning a plug body of a medical device according to any one of embodiments 24 to 37 within a fistula tract; and securing the plug body within the fistula tract.

39. The method of embodiment 38, wherein said securing comprises:
attaching the second suture portion to patient tissue at or near a primary opening of the fistula.

40. The method of embodiment 38, wherein said securing comprises:
securing the first suture portion to the second suture end portion so as to together extend in a path around a volume of tissue between a primary opening and a secondary opening of the fistula.

It is intended that the foregoing detailed description be regarded as illustrative rather than limiting, and that it be understood that it is the following claims, including all equivalents, that are intended to define the spirit and scope of this invention. All publications and patent applications cited in this specification are herein incorporated by reference as if each individual publication or patent application were specifically and individually indicated to be incorporated by reference. Further, any theory, mechanism of operation, proof, or finding stated herein is meant to further enhance understanding of the present invention, and is not intended to limit the present invention in any way to such theory, mechanism of operation, proof, or finding. While the invention has been illustrated and described in detail in the drawings and foregoing description, the same is to be considered as illustrative and not restrictive in character, it being understood that only selected embodiments have been shown and described and that all equivalents, changes, and modifications that come within the spirit of the inventions as defined herein or by the following claims are desired to be protected.

What is claimed is:

1. A medical device for treating a fistula in a patient, the fistula having a fistula tract extending between a primary opening and a secondary opening, the patient having a volume of tissue around which extends a path between the primary opening and the secondary opening, the medical device comprising:
a plug body configured for receipt within the fistula tract; and
an elongate member attached to a first location along the length of the plug body and configured to form a looped assembly extending through the fistula tract and along the path, wherein the looped assembly includes a length of the plug body that is free of the elongate member and/or free of support against tension by the elongate member.

2. The medical device of claim 1, wherein the elongate member comprises a first segment configured to span the path between the primary opening and the second opening.

3. The medical device of claim 1, wherein the plug body comprises an enlarged end having a diameter greater than a diameter of the plug body at a central portion.

4. The medical device of claim 1, wherein:
the plug body includes a sealing member configured to contact patient tissue around the primary opening.

5. The medical device of claim 1, wherein the plug body comprises an absorbable material, preferably wherein the absorbable material comprises an extracellular matrix material or a synthetic material.

6. The medical device of claim 1, wherein the elongate member comprises a biodegradable material, preferably wherein the biodegradable material is configured to persist for at least 30 days after implantation.

7. The medical device of claim 1, wherein the elongate member comprises a looped portion.

8. The medical device of claim 7, wherein a second location along the length of the plug body is configured to engage the looped portion.

9. The medical device of claim 1, comprising an attachment member attached to a second location along the length of the plug body, the second location spaced from the first location, and wherein the attachment member is configured to engage the elongate member to form the looped assembly.

10. The medical device of claim 1, wherein the attachment member and the elongate member comprise biodegradable suture material.

11. A method of treating a fistula, the fistula having a fistula tract extending between a primary opening and a secondary opening, the patient having a volume of tissue around which extends a path between the primary opening and the secondary opening, the method comprising:
inserting a plug body into the fistula, wherein an elongate member is attached to a first location along the length of the plug body; and
securing the plug body within the fistula by forming a looped assembly with the plug body and the elongate member, the looped assembly extending through the fistula tract and along the path, wherein the looped assembly includes a length of the plug body that is free of the elongate member and/or free of support against tension by the elongate member.

12. The method of claim 11, wherein said inserting comprises pulling the plug body into the fistula tract using the elongate member.

13. The method of claim 11, wherein the plug body comprises an enlarged end having a diameter greater than a diameter of the plug body at a central portion, and wherein said inserting causes the enlarged end to become wedged into the primary opening.

14. The method of claim 11, comprising
positioning a sealing member of the plug body against patient tissue around the primary opening.

15. The method of claim 11, wherein the elongate member comprises a looped portion.

16. A medical device for treating a fistula in a patient, the medical device comprising:
a plug body configured for receipt within a fistula tract of the fistula, the plug body comprising a plurality of sheets of biocompatible material, wherein the plug body includes a longitudinally extending core in which a first set of longitudinally extending portions of the sheets are fixed relative to one another, and wherein the plug body further includes a plurality of radial fin portions provided by a second set of longitudinally extending portions of the sheets that are movable relative to one another and positioned or positionable to extend radially outwardly in different directions from the longitudinally extending core of the plug body.

17. The medical device of claim 16, wherein:
the sheets are positioned in a stacked configuration; and
the longitudinally extending core comprises suture material affixing the first set of longitudinally extending portions of the sheets to one another.

18. The medical device of claim 17, wherein the suture material includes at least one suture line comprising a suture extending from a leading end of the plug body to a trailing end of the plug body.

19. The medical device of claim 16, wherein longitudinally extending side edges of the radial fin portions define a tapering width of the plug body, optionally wherein the width of the plug body increases in a direction from a leading end of the plug body to a trailing end of the plug body.

20. The medical device of claim 16, comprising a first suture portion extending beyond a leading end of the plug body, wherein the first suture portion and the second suture portion are configured to extend around a volume of tissue around which extends a path between a primary opening and a secondary opening of the fistula.
